(19) United States
(12) Patent Application Publication
(10) Pub. No.: US 2004/0136931 A1
(43) Pub. Date: Jul. 15, 2004

(54) Photoprotective cosmetic compositions comprising 3-(2-azacycloalkylidene)-1,3-dihydroindol-2-one compounds

(75) Inventor: Roger Rozot, Lagny/Marne (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL S.A., Paris (FR)

(21) Appl. No.: 10/720,163

(22) Filed: Nov. 25, 2003

Related U.S. Application Data
(60) Provisional application No. 60/449,611, filed on Feb. 26, 2003.

(30) Foreign Application Priority Data
Nov. 29, 2002 (FR) 02/15054

(51) Int. Cl. A61K 7/42; C 07D 4 3/04

(52) U.S. Cl. 424/59; 548/465

(57) ABSTRACT

Topically applicable cosmetic/dermatological sunscreen compositions well suited for the UV-photoprotection of human skin and/or hair, contain (a) an effective UV-screening amount of at least one 3-(2-azacycloalkylidene)-1,3-dihydroindol-2-one compound having the structural formula (I):

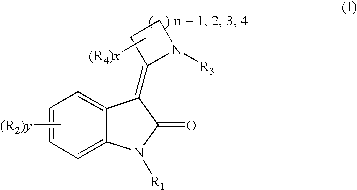

formulated into (b) topically applicable, cosmetically/dermatologically acceptable carrier therefor.

### CROSS-REFERENCE TO PRIORITY/PROVISIONAL APPLICATIONS

This application claims priority under 35 U.S.C. § 119 of FR-02/15054, filed Nov. 29, 2002, and of provisional application Serial No. 60/449,611, filed Feb. 26, 2003, both hereby expressly incorporated by reference. This application is also a continuation of said '611 provisional.

### CROSS-REFERENCE TO COMPANION APPLICATION

Copending application Ser. No. ______ [Attorney Docket No. 016800-582], filed concurrently herewith and assigned to the assignee hereof.

### BACKGROUND OF THE INVENTION

1. Technical Field of the Invention

The present invention relates to cosmetic or dermatological compositions suited for photoprotecting the skin and/or the hair against UV radiation, in particular solar radiation, which comprise an effective quantity of at least one 3-(2-azacycloalkylidene)-1,3-dihydroindol-2-one compound having a particular chemical structure.

The invention also relates to the formulation of photoprotective cosmetic or dermatological compositions, from these 3-(2-azacycloalkylidene)-1,3,dihydroindol-2-one compounds, in particular as sunscreens which are active in the UV radiation domain.

2. Description of Background/Related/Prior Art

It is known that radiation having wavelengths of between 280 nm and 400 nm allows tanning of the human epidermis, and that radiation having wavelengths more particularly of between 280 nm and 320 nm, known by the name UV-B radiation, causes erythemas and skin burns which can impede the development of natural tanning. For these reasons, and for aesthetic reasons, there is an increasing demand for means for controlling this natural tanning in order to control the color of the skin. It is therefore advisable to screen out this UV-B radiation.

It is also known that UV-A rays having wavelengths of between 320 nm and 400 nm, which cause tanning of the skin, are capable of inducing its impairment, in particular in the case of a sensitive skin or of a skin continually exposed to solar radiation. UV-A rays cause in particular a loss of elasticity of the skin and the appearance of wrinkles which lead to premature skin aging. They promote the onset of the erythematous reaction or amplify this reaction in some subjects and may even be responsible for phototoxic or photoallergic reactions. Thus, for aesthetic and cosmetic reasons such as the preservation of the natural elasticity of the skin, for example, an increasing number of people wish to control the effect of UV-A rays on their skin. It is therefore desirable also to screen out UV-A radiation.

Numerous compounds intended for photoprotecting (UV-A and/or UV-B) the skin are known to this art.

Most are aromatic compounds which absorb UV radiation in the region between 280 nm and 315 nm, or in the region between 350 nm and 400 nm, or in the whole of these two regions. They are most often formulated in anti-sun compositions which are provided in the form of an oil-in-water type emulsion (that is to say a cosmetically acceptable carrier consisting of an aqueous dispersing continuous phase and an oily dispersed discontinuous phase) and which therefore contain, in various concentrations, one or more lipophilic and/or hydrophilic conventional organic screening agents having an aromatic functional group, which are capable of selectively absorbing harmful UV radiation, these screening agents (and their quantities) being selected according to the desired sun protection factor (the sun protection factor being mathematically expressed by the ratio of the irradiation time necessary to reach the erythematogenetic threshold with the UV screening agent to the time necessary to reach the erythematogenetic threshold without the UV screening agent).

In addition to their screening power, these compounds with anti-UV activity should also have good cosmetic properties in the compositions containing them, a good solubility in the customary solvents and in particular fatty substances such as oils and fats, and a good stability to UV radiation (photostability).

### SUMMARY OF THE INVENTION

It has now surprisingly and unexpectedly been determined that a certain family of organic UV screening agents of the 3-(2-azacycloalkylidene)-1,3-dihydroindol-2-one compound type which lack photo-oxidizing activity and which exhibit, in addition to excellent screening properties in the UV-A and/or UV-B radiation domain, a very good solubility in the customary organic solvents and in particular fatty substances such as oils, an excellent photostability and excellent cosmetic properties, making them particularly appropriate for use as sun screening agents in cosmetic compositions intended for protecting the skin and/or the hair against ultraviolet radiation.

### DETAILED DESCRIPTION OF BEST MODE AND SPECIFIC/PREFERRED EMBODIMENTS OF THE INVENTION

The present invention thus features cosmetic compositions for topical application, in particular for photoprotecting the skin and/or the hair, which comprise, in a cosmetically acceptabie carrier, at least one 3-(2-azacycloalkylidene)-1,3-dihydroindol-2-one compound corresponding to the following formula (I)

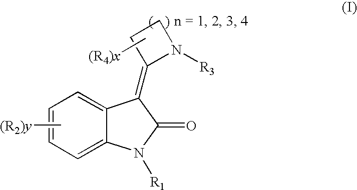

in which R₁ and R₃, which may be identical or different, are each a hydrogen atom, a saturated or unsaturated, linear or branched, C₁-C₂₂ alkyl radical optionally substituted with one or more groups A₁, a saturated or unsaturated, 4-to 7-atom ring member optionally containing at least one heteroatom selected from among N, O and S, optionally fused with another ring, optionally substituted with one or more groups A₁, with the proviso that these rings may contain one or more carbonyl or thiocarbonyl functional groups, one of the groups C(═NR₅)R′₅, C(═NR₅)NR′₅R″₅, COR₅, CSR₅, COOR₅, CONR₅R′₅, CSNR₅R′₅, SO₂R₅, and SO₂NR₅R′₅ wherein R₅, R′₅ and R″₅, which may be identical or different, are each hydrogen, a linear or branched C₁-C₂₂ alkyl radical or a 4- to 7-atom ring member optionally containing at least one heteroatom selected from among N, O, S, optionally fused with another ring, with the proviso that these rings may contain one or more carbonyl or thiocarbonyl functional groups, the alkyl radical of said rings being saturated or unsaturated and optionally substituted with at least one substituent A₂; R₂ and R₄, which may be identical or different, are each a hydrogen atom, a saturated or unsaturated, linear or branched C₁-C₂₂ alkyl radical optionally substituted with one or more groups A₁, a saturated or unsaturated, 4- to 7-atom ring member optionally containing at least one heteroatom selected from among N, O and S, optionally substituted with one or more groups A₁, optionally fused with another ring or with another 3-(2-azacycloalkylidene)-1,3-dihydroindol-2-one according to this invention, with the proviso that these rings may contain one or more carbonyl or thiocarbonyl functional groups, optionally positively charged like imidazolium, pyridinium, pyrazolium, triazolium, a halogen such as F, Cl, Br, one of the groups CF₃, CN, OR₅, SR₅, NR₅R′₅, C(═NR₅)R′₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅, and NR₅R′₅R″₅R′″₅⁺ wherein R₅, R′₅, R″₅ and R′″₅, which may be identical or different, are each hydrogen, a linear or branched C₁-C₂₂ alkyl radical or a 4- to 7- atom ring member which may contain at least one heteroatom selected from among N, O, S, optionally fused with another ring, with the proviso that these rings may contain one or more carbonyl or thiocarbonyl functional groups, the alkyl radical or the said rings being saturated or unsaturated and optionally substituted with at least one substituent A₂; A₁ is a halogen such as F, Cl, Br, a saturated or unsaturated, linear or branched C₁-C₂₂ alkyl radical, optionally substituted with one or more groups A₂, one of the groups CF₃, CN, OR₅, SR₅, NR₅R′₅, C(═NR₅)R′₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅, SiR₅R′₅R″₅, SiR₅(OSiR′₅R″₅R′″₅)OSiR′₅R″₅R′″₅, and NR₅R′₅R″₅R′″₅ + wherein R₅, R′₅, R″₅ and R′″₅, which may be identical or different, are each hydrogen, a linear or branched C₁-C₂₂ alkyl radical or a 4- to 7- atom ring member which may contain at least one heteroatom selected from among N, O, S, optionally fused with another ring, with the proviso that these rings may contain one or more carbonyl or thiocarbonyl functional groups, the alkyl radical or said rings being saturated or unsaturated and optionally substituted with at least one substituent A₂, a saturated or unsaturated 4- to 7-atom ring member optionally containing at least one heteroatom selected from among N, O and S, optionally substituted with one or more groups A₂, optionally fused with another ring or with another 3(2-azacycloalkylidene)-1,3-dihydroindol-2-one according to this invention, with the proviso that these rings may contain one or more carbonyl or thiocarbonyl functional groups, optionally positively charged like imidazolium pyridinium, pyrazolium, triazolium; A₂ is a halogen such as F, Cl, Br, a saturated or unsaturated, linear or branched C₁-C₂₂ alkyl radical, one of the groups CF₃, CN, OR, SR, NRR′, C(═NR)R′, C(═NR)NR′R″, NRC(═NR′)NR″R′″, COR, CSR, COOR, CONRR′, NRCOR′, NRCONR′R″, CSNRR′, SO₂NRR′, NRSO₂R′, SO₂R, SiRR′R″, SiR(OSiR′R″R′″)OSiR′R″R′″, and NRR′R″R′″+ wherein R, R′, R″ and R′″, which may be identical or different, are each hydrogen or a linear or branched C₁-C₂₂ alkyl radical; y is 1, 2, 3 or 4; and x ranges from 1 to 2n+2.

As examples of saturated or unsaturated, linear or branched C₁-C₂₂ alkyl radicals there may be mentioned: methyl, isopropyl, 2-ethylhexyl, tert-butyl, ethylene, propylene. This list is not limiting.

As examples of heterocycles, there may be mentioned: pyrrole, furan, thiophene, imidazole, oxazole, thiazole, pyrazole, isoxazole, isothiazole, triazole, oxadiazole, thiadiazole, tetrazole, pyridine, piperidine, pyrimidine, piperazine, pyridazine, pyrazine, triazine, morpholine, pyrrolidine, thiazolidine. This list is not limiting.

As examples of saturated or unsaturated 4-, 5-, 6- or 7-atom carbon rings, there may be mentioned: cyclobutyl, cyclopentyl, cyclohexyl, cyclohexenyl, phenyl or cycloheptyl. This list is not limiting.

Certain 3-(2-azacycloalkylidene)-1,3-dihydroindol-2-one derivatives of formula (I) in accordance with the invention are known in the chemical literature and in particular certain of them have been described in the articles in Indian Journal of Chemistry (1983), 22B, 1083-1086 and in Journal of Medicinal Chemistry (1989), 32, 437 -444.

The 3-(2-azacycloalkylidene)-1,3-dihydroindol-2-one compounds of formula (I) in accordance with the invention may be prepared according to a method of synthesis using an acetal of lactam and a 1,3-dihydroindol-2-one derivative at room temperature in anhydrous ether according to the following reaction scheme cited in the above articles:

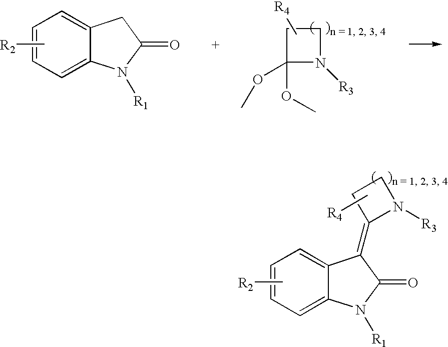

Numerous 1,3-dihydroindol-2-one and lactam derivatives are available from most suppliers of chemical products, for example:

| 1,3-dihydroindol-2- | | | |
|---|---|---|---|
| one Derivative | Supplier | Reference | CAS |
| 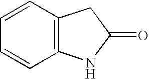 | ABCR | AV18764 | 59-48-3 |
| 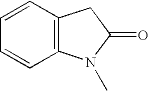 | Maybridge | RH 01320 | 61-70-1 |
| 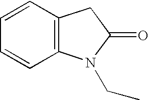 | MicroChemistry Ltd. | 39248 | 61-28-9 |
| 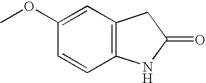 | Specs | AK 830/25033062 | |
| 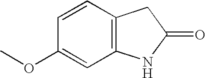 | Finoraga SA | 000048 | |
| 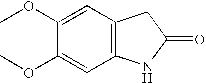 | Maybridge | SEW 04570 | 6286-64-2 |
| 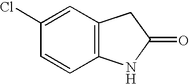 | Aldrich | 12,748-5 | 17630-75-0 |
| 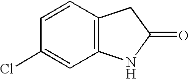 | ABCR | AV9674 | 56341-37-8 |
| 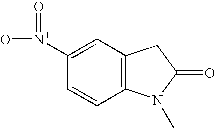 | Maybridge | RH 01323 | 20870-89-7 |
| 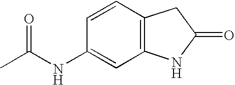 | MDPI | 11566 | |
| 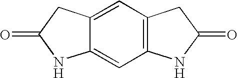 | MDPI | 11349 | |
| 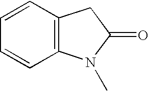 | Specs | AC- 907/25005257 | |
| 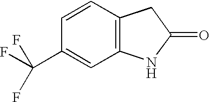 | Butt Park Ltd. | 24/07-32 | |
| Lactam Derivative | Supplier | Reference | CAS |
| 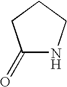 | ABCR | AV15332 | 616-45-5 |
| 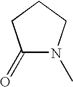 | ABCR | AV12260 | 872-50-4 |
| 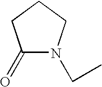 | ABCR | 1549765 | 2687-91-4 |
| 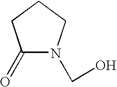 | Salor | S36,255-7 | 15438-71-8 |
| 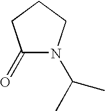 | BASF-Misc. | | 3772-26-7 |
| 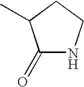 | Lancaster | | 2555-05-7 |
| 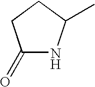 | Aldrich | M7,970-0 | 108-27-0 |
| 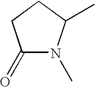 | Aldrich | D18,410-1 | 5075-92-3 |
| 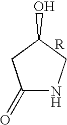 | Aldrich | 47,916-0 | 22677-21-0 |
| 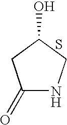 | Aldrich | 47,917-9 | 68108-18-9 |
| 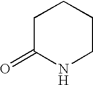 | ABCR | AV12271 | 675-20-7 |
| 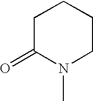 | Advan-Synth | 001427 | 931-20-4 |
| 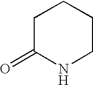 | Maybridge | BTBG 00104 | |
| 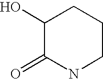 | Salor | S43,760-3 | 19365-08-3 |
| 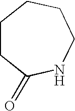 | ABCR | AV19374 | 105-60-2 |
| 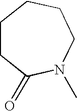 | ABCR | AV17776 | 2556-73-2 |
| 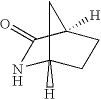 | Acros | 29704-0010 | 79200-56-9 |
| 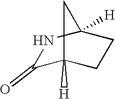 | Acros | 29705-0010 | 130931-83-8 |
| 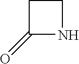 | Aldrich | 32,846-4 | 930-21-2 |
| 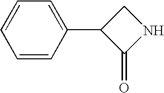 | Salor | S90,675-1 | 17197-57-8 |
| 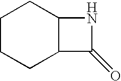 | Acros | 33584-0010 | 34102-49-3 |
| 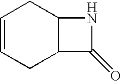 | Maybridge | JFD 02755 | |
| 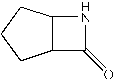 | Acros | 33583-5000 | 22031-52-3 |

The acetals of lactam may be obtained from lactams according to the following reaction scheme disclosed in the articles Tetrahedron letters (1994), 35 (18), 2951-2954 and Journal of Organic Chemistry (1984), 49, 3659-3660:

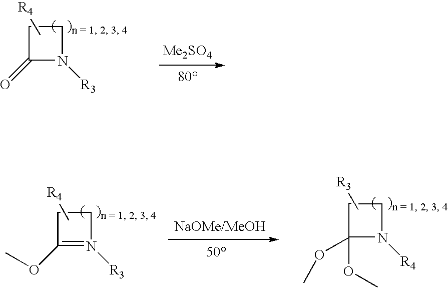

Depending on the starting lactam, steps for protecting and then deprotecting certain functional groups such as OH or NH, well known to those skilled in the art, may be necessary.

Among the preferred compounds of formula (I), there may be mentioned those of the following formulae (II) or (III):

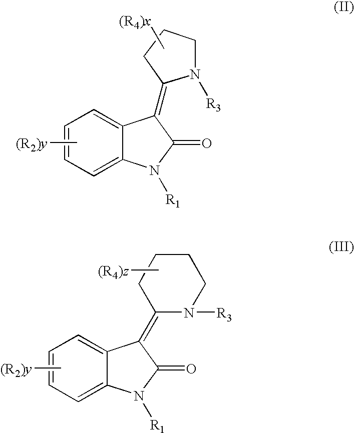

in which R₁ and R₃, which may be identical or different, are each hydrogen, a saturated or unsaturated, linear or branched C₁-C₈ alkyl radical, a phenyl radical, or a radical COR₅ or SO₂R₅ wherein R₅ is a saturated or unsaturated, linear or branched C₁-C₈ alkyl radical, or a phenyl radical; R₂ is hydrogen, a saturated or unsaturated, linear or branched C₁-C₈ alkyl radical, a phenyl radical, one of the radicals OR₅, NR₅′₅, NR₅COR′₅, COOR₅ and CONR₅R′₅ wherein R₅ and R′₅, which may be identical or different, are each hydrogen, a saturated or unsaturated, linear or branched C₁-C₈ alkyl radical, a phenyl radical, or the radical CF₃; R₄ is hydrogen, a saturated or unsaturated, linear or branched C₁-C₈ alkyl radical, a phenyl radical or a radical OR₅ wherein R₅ is hydrogen, a saturated or unsaturated, linear or branched C₁-C₈ alkyl radical, or a phenyl radical; and x, y and z are each 1, 2, 3 or 4.

Among the 3-(2-azacycloalkylidene)-1,3-dihydroindol-2-one derivatives of formula (I), there may be mentioned still more particularly those of formula (II) where:

R₂ is hydrogen, OR₅, NR₅COR′₅ or NR₅R′₅ wherein R₅ and R′₅, which may be identical or different, are each hydrogen, a saturated or unsaturated, linear or branched C₁-C₈ alkyl radical, or a phenyl radical;

R₄ is hydrogen;

R₁ and R₃, which may be identical or different, are each hydrogen, a saturated or unsaturated, linear or branched C₁-C₈ alkyl radical, or a phenyl radical and still more particularly those described in the following table, which are, commercially available from Specs/Biospecs:

| | | Name (Specs/Biospecs |
|---|---|---|
| Compound No. | Formula | Reference) |
| 1 | 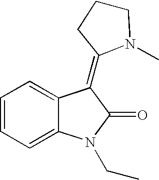 | 1-ethyl-3-(1- methylpyrrolidin-2- ylidene)-1,3- dihydroindol-2-one (AH- 262/33341016) |
| 2 | 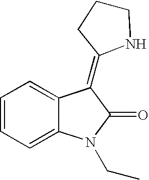 | 1-ethyl-3-pyrrolidin-2- ylidene-1,3- dihydroindol-2-one (AH- 262/33341015) |
| 3 | 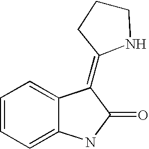 | 3-pyrrolidin-2-ylidene- 1,3-dihydroindol-2-one (AH-262/33341017) |

The compounds of formula (I) are generally present in the composition of the invention in proportions of between 0.01% and 20% by weight, preferably between 0.1% and 10% by weight, relative to the total weight of the composition.

The compositions in accordance with the invention may for the main comprise other additional organic or inorganic UV-screening agents active in UVA and/or UVB which are water-soluble or fat-soluble or insoluble in commonly used cosmetic solvents.

The additional organic screening agents are chosen in particular from anthranilates; cinnamic derivatives; dibenzoylmethane derivatives; salicylic derivatives, camphor derivatives; triazine derivatives such as those described in U.S. Pat. No. 4,367,390, EP-863,145, EP-517,104, EP-570,838, EP-796,851, EP-775,698, EP-878,469, EP-933,376, EP-507,691, EP-507,692, EP-790,243 and EP-944,624; benzophenone derivatives, β,β-diphenyl acrylate derivatives, benzotriazole derivatives, benzamalonate derivatives, benzirnidazole derivatives; imidazolines; bis-benzoazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2-303,549, DE-1-9-726,184 and EP-893,119; benzoxazole derivatives as described in EP-0-832,642, EP-1-027,883, EP-1-300,137 and DE-1-0-162,844 screening polymers and screening silicones such as those described in particular in WO 93/04665; dimers derived from α-alkylstyrene such as those described in DE-1-9-855,649; 4,4-diarylbutadienes such as those described in EP-0-967,200, DE-1-9-746,654, DE-1-9-755,649, EP-A-1-008,586, EP-1-133,980 and EP-133,981 and mixtures thereof.

As examples of additional organic screening agents, there may be mentioned those designated below under their INCI name:

Para-Aminobenzoic Acid Derivatives:

PABA,

Ethyl PABA,

Ethyl Dihydroxypropyl PABA,

Ethylhexyl Dimethyl PABA sold in particular under the name “ESCALOL 507” by ISP,

Glyceryl PABA,

PEG-25 PABA sold under the name “UVINUL P25” by BASF,

Salicylic Derivatives:

Homosalate sold under the name “Eusolex HMS” by Rona/EM Industries,

Ethylhexyl Salicylate sold under the name “NEO HELIOPAN OS” by Haarmann and REIMER,

Dipropyleneglycol Salicylate sold under the name “DIPSAL” by SCHER,

TEA Salicylate, sold under the name “NEO HELIOPAN TS” by Haarmann and REIMER,

Dibenzoylmethane Derivatives:

Butyl Methoxydibenzoylmethane sold in particular under the trademark “PARSOL 1789” by HOFFMANN LA ROCHE, Isopropyl Dibenzoylmethane,

Cinnamic derivatives:

Ethylhexyl Methoxycinnamate sold in particular under the trademark “PARSOL MCX” by HOFFMANN LA ROCHE,

Isopropyl Methoxy cinnamate,

Isoamyl Methoxy cinnamate sold under the trademark “NEO HELIOPAN E 1000” by HAARMANN and REIMER,

Cinoxate,

DEA Methoxycinnamate,

Diisopropyl Methylcinnamate,

Glyceryl Ethylhexanoate Dimethoxycinnamate,

β,β′-Diphenyl Acrylate Derivatives:

Octocrylene sold in particular under the trademark “UVINUL N539” by BASF,

Etocrylene, sold in particular under the trademark “UVINUL N35” by BASF,

Benzophenone Derivatives:

Benzophenone-1 sold under the trademark “UVINUL 400” by BASF,

Benzophenone-2 sold under the trademark “UVINUL D50” by BASF,

Benzophenone-3 or Oxybenzone, sold under the trademark “UVINUL M40” by BASF,

Benzophenone-4 sold under the trademark “UVINUL MS40” by BASF,

Benzophenone-5,

Benzophenone-6 sold under the trademark “Helisorb 11” by Norquay,

Benzophenone-8 sold under the trademark “Spectra-Sorb UV-24” by American Cyanamid,

Benzophenone-9 sold under the trademark “UVINUL DS-49” by BASF,

Benzophenone-12,

n-Hexyl 2-(4-diethylamino-2-hydroxybenzoyl)benzoate,

Benzylidenecamphor Derivatives:

3-Benzylidene camphor manufactured under the name “MEXORYL SD” by CHIMEX,

4-Methylbenzylidene camphor sold under the name “EUSOLEX 6300” by MERCK,

Benzylidene Camphor Sulphonic Acid manufactured under the name “MEXORYL SL” by CHIMEX,

Camphor Benzalkonium Methosulphate manufactured under the name “MEXORYL SO” by CHIMEX,

Terephthalylidene Dicamphor Sulphonic Acid manufactured under the name “MEXORYL SX” by CHIMEX,

Polyacrylamidomethyl Benzylidene Camphor manufactured under the name “MEXORYL SW” by CHIMEX,

Phenylbenzimidazole Derivatives:

Phenylbenzimidazole Sulphonic Acid sold in particular under the trademark “EUSOLEX 232” by MERCK,

Disodium Phenyl Dibenzimidazole Tetra-sulphonate sold under the trademark “NEO HELIOPAN AP” by Haarmann and REIMER,

Triazine Derivatives:

Anisotriazine sold under the trademark “TINOSORB S” by CIBA GEIGY,

Ethylhexyl triazone sold in particular under the trademark “UVINUL T150” by BASF,

2,4,6-Tris(diisobutyl 4′-aminobenzalmalonate)-s-triazine,

Diethylhexyl Butamido Triazone sold under the trademark “UVASORB HEB” by SIGMA 3V,

Phenylbenzotriazole Derivatives:

Drometrizole Trisiloxane sold under the name “Silatrizole” by RHODIA CHIMIE,

Methylene bis-Benzotriazoiyl Tetramethylbutylphenol, sold in solid form under the trademark “MIXXIM BB/100” by FAIRMOUNT CHEMICAL or in micronized form in aqueous dispersion under the trademark “TINOSORB M” by CIBA SPECIALTY CHEMICALS,

Anthranilic Derivatives:

Menthyl anthranilate sold under the trademark “NEO HELIOPAN MA” by Haarmann and REIMER,

Imidazoline Derivatives:

Ethylhexyl Dimethoxybenzylidene Dioxoimidazoline Propionate,

Benzalmalonate Derivatives:

Polyorganosiloxane with benzalmalonate functional groups sold as the product Polysilicone-15 sold under the trademark “PARSOL SLX” by HOFFMANN LA ROCHE,

4,4-diarylbutadiene Derivatives:

1,1-dicarboxy(2,2′-dimethylpropyl)-4,4-diphenyl-butadiene

Benzoxazole Derivatives:

2,4-bis-[5-1(dimethylpropyl)benzoxazol-2-yl-(4-phenyl)-imino]-6-(2-ethylhexyl)-imino-1,3,5-triazine sold under the trademark Uvasorb K2A by Sigma 3V; and mixtures thereof.

The additional organic UV-screening agents which are preferred are chosen from the following compounds:

Ethylhexyl Salicylate,

Ethylhexyl Methoxycinnamate,

Octocrylene,

Phenylbenzimidazole Sulphonic Acid,

Benzophenone-3,

Benzophenone-4,

Benzophenone-5,

n-Hexyl 2-(4-diethylamino-2-hydroxybenzoyl)benzoate, 4-Methylbenzylidene camphor,

Terephthalylidene dicamphor sulfonic acid,

Disodium Phenyl Dibenzimidazole Tetra-sulphonate,

2,4,6-Tris(diisobutyl 4′-aminobenzalmalonate)-s-triazine,

Anisotriazine,

Ethylhexyl triazone,

Diethylhexyl Butamido Triazone,

Methylene bis-Benzotriazolyl

Tetramethylbutylphenol,

Drometrizole Trisiloxane,

Polysilicone-15

1,1-dicarboxy(2,2′-dimethylpropyl)-4,4-diphenylbutadiene 2,4-bis-[5-1(dimethylpropyl)benzoxazol-2-yl-(4-phenyl)-imino]-6-(2-ethylhexyl)-imino-1,3,5-triazine and mixtures thereof.

The additional inorganic screening agents are chosen from pigments or alternatively nanopigments (mean primary particle size: generally between 5 nm and 100 nm, preferably between 10 nm and 50 nm) of metal oxides, coated or otherwise, such as for example nanopigments of titanium oxide (amorphous or crystallized in rutile and/or anatase from), of iron oxide, of zinc oxide, of zirconium oxide or of cerium oxide which are all UV photoprotective agents well known per se. Conventional coating agents are moreover alumina and/or aluminium stearate. Such coated or uncoated nanopigments of metal oxides are described in particular in EP-518,772 and EP-518,773.

The additional UV-screening agents in accordance with the invention are generally present in the compositions according to the invention in proportions ranging from 0.01% to 20% by weight relative to the total weight of the composition, and preferably ranging from 0.1% to 10% by weight relative to the total weight of the composition.

The cosmetic compositions according to the invention may contain, in addition, agents for artificially bronzing and/or tanning the skin (self-tanning agents) such as dihydroxyacetone (DHA).

The compositions in accordance with the present invention may comprise, in addition, conventional cosmetic adjuvants chosen in particular from fatty substances, organic solvents, ionic or nonionic thickeners, demulcents, humectants, antioxidants, moisturizers, desquamating agents, anti-free-radical agents, antipollution agents, antibacterials, anti-inflammatory agents, depigmenting agents, propigmenting agents, opacifiers, stabilizers, emollients, silicones, anti-foaming agents, insect repellents, perfumes, preservatives, anionic, cationic, nonionic, zwitterionic or amphoteric surfactants, substance P antagonists, substance CGRP antagonists, fillers, pigments, polymers, propellants, alkalinizing or acidifying agents or any other ingredient normally used in the cosmetic and/or dermatological field.

The fatty substances may consist of an oil or a wax or mixtures thereof. The expression oil is understood to mean a compound which is liquid at room temperature. The expression wax is understood to mean a compound which is solid or substantially solid at room temperature, and whose melting point is generally greater than 35° C.

As oils, there may be mentioned mineral oils (paraffin); vegetable oils (sweet almond, macadamia, blackcurrant seed or jojoba oil); synthetic oils such as perhydrosqualene, fatty alcohols, acids or esters (such as C₁₂-C₁₅ alcohol benzoate sold under the trademark “Finsolv TN” by WITCO, octyl palmitate, isopropyl lanolate, triglycerides including those of capric/caprylic acids), oxyethylenated or oxypropylenated fatty esters and ethers; silicone oils (cyclomethicone, polydimethylsiloxanes or PDMS) or fluorinated oils, polyalkylenes.

As waxy compounds, there may be mentioned paraffin, carnauba wax, beeswax, hydrogenated castor oil.

Among the organic solvents, there may be mentioned lower alcohols and polyols. The latter may be chosen from glycols and glycol ethers such as ethylene glycol, propylene glycol, butylene glycol, dipropylene glycol or diethylene glycol.

The thickeners may be chosen in particular from crosslinked acrylic polymers which are Carbomers, crosslinked acrylate/C₁₀-C₃₀ alkyl acrylate polymers of the Pemulen or polyacrylate-3 type sold under the name VISCOPHOBE DB 1000 by Amerchol; polyacrylamides such as polyacrylamide, C₁₃-C₁₄ isoparaffin and laureth-7 emulsions sold under the name SEPIGEL 305 by SEPPIC, homopolymers or copolymers of AMPS such as HOSTACERIN AMPS sold by CLARIANT, guar gums and modified or unmodified celluloses such as hydroxypropylated guar gum, methylhydroxyethylcellulose and hydroxypropylmethylcellulose, xanthan gum, nanometric silicas of the Aerosil type.

Of course, those skilled in the art will be careful to choose the possible additional compound or compounds cited above and/or their quantities such that the advantageous properties intrinsically attached to the compounds in accordance with the invention are not, or not substantially, impaired by the addition(s) envisaged.

The compositions according to the invention may be prepared according to techniques well known to persons skilled in the art, in particular those intended for the preparation of oil-in-water or water-in-oil type emulsions.

This composition may be provided in particular in the form of a simple or complex emulsion (O/W, W/O, O/W/O or W/O/W) such as a cream or a milk, in the form of a gel or a gel cream, or in the form of a lotion, an oil, a powder or a solid stick, and may be optionally packaged as an aerosol and may be provided in the form of a mousse or a spray.

Preferably, the compositions according to the invention are provided in the form of an oil-in-water or water-in-oil emulsion.

When it is an emulsion, the aqueous phase thereof may comprise a nonionic vesicular dispersion prepared according to known methods (Bangham, Standish and Watkins. J. Mol. Biol. 13, 238 (1965), FR-2-315,991 and FR-2-416,008).

When the cosmetic composition according to the invention is used for the care of the human epidermis, it may be provided in the form of a suspension or a dispersion in solvents or fatty substances, in the form of a nonionic vesicular dispersion or in the form of an emulsion, preferably of the oil-in-water type, such as a cream or a milk, in the form of an ointment, a gel, a gel cream, an anti-sun oil, a solid stick, a powder, an aerosol mousse or a spray.

When the cosmetic composition according to the invention is used for hair care, it may be provided in the form of a shampoo, a lotion, a gel, an emulsion, a nonionic vesicular dispersion and may constitute, for example, a rinse-out composition to be applied before or after shampooing, before or after dyeing or bleaching, or before, during or after permanent waving or hair straightening, a hair styling or treatment lotion or gel, a blow drying or hair setting lotion or gel, a composition for permanent waving or straightening, dyeing or bleaching the hair.

When the composition is used as a make-up product for the nails, the lips, the eyelashes, the eyebrows or the skin, such as a treatment cream for the epidermis, a foundation, a lipstick, an eyeshadow, a blusher, a mascara or an eyeliner, it may be provided in an anhydrous or aqueous, solid or pasty form, such as oil-in-water or water-in-oil emulsions, nonionic vesicular dispersions or suspensions.

As a guide, for the anti-sun formulations in accordance with the invention which have a carrier of the oil-in-water emulsion type, the aqueous phase (comprising in particular the hydrophilic screening agents) generally represents from 50% to 95% by weight, preferably from 70% to 90% by weight, relative to the whole formulation, the oily phase (comprising in particular the lipophilic screening agents) from 5% to 50% by weight, preferably from 10% to 30% by weight, relative to the whole formulation, and the (co)emulsifier(s) from 0.5% to 20% by weight, preferably from 2% to 10% by weight, relative to the whole formulation.

Another subject of the invention is the use of a 3-(2-azacycloalkylidene)-1,3-dihydroindol-2-one derivative of formula (I) as defined above in a cosmetic or dermatological composition as UV radiation screening agent.

Another subject of the invention is the use of a 3-(2-azacycloalkylidene)-1,3-dihydroindol-2-one derivative of formula (I) as defined above in a cosmetic composition as an agent for controlling the variation in the color of the skin due to UV radiation.

Another subject of the invention is the use of a 3-(2-azacycloalkylidene)-1,3-dihydroindol-2-one derivative of formula (I) as defined above as an agent which photostabilizes synthetic polymers such as plastics or glasses, in particular spectacle glasses or contact lenses.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### 1-Ethyl-3-(1-methylpyrrolidin-2-ylidene)-1,3-dihydroindol-2-one (compound 1)

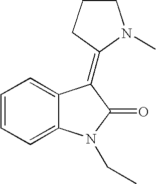

This compound may be bought from Specs/Biospecs under the reference AH-262/33341016 or may be obtained according to the method of synthesis defined above from 1-ethyl-1,3-dihydroindol-2-one (cas: 61-28-9) and from 1-methylpyrrolidin-2-one (cas: 872-50-4).

This compound has the following sun screening properties:

λmax=350 nm (92.8% acetonitrile+7.2% DMSO)

εmax=17501 1.mol⁻¹.cm⁻¹

E1%=722.

This compound is photostable as the following experiment shows: After one hour of irradiation in a solar simulator (UVA=18.6 mW/cm², UVB=0.6 mW/cm²) of an oily film containing 0.5% of this compound, the residual level of the said compound is 92%.

RPE (Resonance Paramagnetic Electronic) studies show that this compound does not manifest any pro-oxidizing activity in a formulated medium under irradiation under the same conditions.

### Example 2

### 1-Ethyl-3-pyrrolidin-2-ylidine-1,3-dihydroindol-2-one (compound 2)

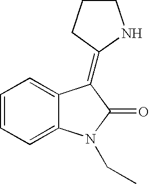

This compound may be bought from Specs/Biospecs under the reference AH-262/33341015 or may be obtained according to the method of synthesis defined above from 1-ethyl-1,3-dihydroindol-2-one (cas: 61-28-9) and from 1-pyrrolidine-2-one (cas: 616-45-5).

This compound has the following sun screening properties:

λmax=326 nm (92.8% acetonitrile+7.2% DMSO)

εmax=22600 1.mol⁻¹.cm⁻¹.

### Example 3

### 3-Pyrrolidin-2-ylidene-1,3-dihydroindol-2-one (compound 3)

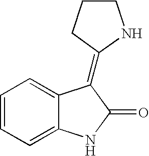

This compound may be bought from Specs/Biospecs under the reference AH-262/33341017 or may be obtained according to the method of pyrrolidine-2-one (cas: 616-45-5).

This compound has the following sun screening properties:

λmax=328 nm (92.8% acetonitrile+7.2% DMSO)

εmax=8100 1.mol⁻¹.cm⁻¹.

### Example of Composition 1

As an example of composition, there may be mentioned an anti-sun lotion comprising the compound of example 1 at the concentration of 0.5% in Miglyol.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/dermatological sunscreen composition suited for the UV-photoprotection of the skin and/or hair, comprising (a) an effective UV-screening amount of at least one 3-(2-azacycloalkylidene)-1,3-dihydroindol-2-one compound having the following structural formula (I): 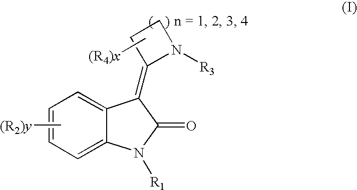
in which R₁ and R₃, which may be identical or different, are each a hydrogen atom, a saturated or unsaturated, linear or branched, C₁-C₂₂ alkyl radical optionally substituted with one or more groups A₁, a saturated or unsaturated, 4-to 7-atom ring member optionally containing at least one heteroatom selected from among N, O and S, optionally fused with another ring, optionally substituted with one or more groups A₁, with the proviso that these rings may contain one or more carbonyl or thiocarbonyl functional groups, one of the groups C(═NR₅)R′₅, C(═NR₅)NR′₅R″₅, COR₅, CSR₅, COOR₅, CONR₅R′₅, CSNR₅R′₅, SO₂R₅, and SO₂NR₅R′₅ wherein R₅, R′₅ and R″₅, which may be identical or different, are each hydrogen, a linear or branched C₁-C₂₂ alkyl radical or a 4- to 7-atom ring member optionally containing at least one heteroatom selected from among N, O, S, optionally fused with another ring, with the proviso that these rings may contain one or more carbonyl or thiocarbonyl functional groups, the alkyl radical of said rings being saturated or unsaturated and optionally substituted with at least one substituent A₂; R₂ and R₄, which may be identical or different, are each a hydrogen atom, a saturated or unsaturated, linear or branched C₁-C₂₂ alkyl radical optionally substituted with one or more groups A₁, a saturated or unsaturated, 4- to 7-atom ring member optionally containing at least one heteroatom selected from among N, O and S, optionally substituted with one or more groups A , optionally fused with another ring or with another 3-(2-azacycloalkylidene)-1,3-dihydroindol-2-one compound, with the proviso that these rings may contain one or more carbonyl or thiocarbonyl functional groups, optionally positively charged, a halogen, one of the groups CF₃, CN, OR₅, SR₅, NR₅R′₅, C(═NR₅)R′₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅, and NR₅R′₅R″₅R′″₅⁺ wherein R₅, R′₅, R″₅ and R′″₅, which may be identical or different, are each hydrogen, a linear or branched C₁-C₂₂ alkyl radical or a 4- to 7- atom ring member which may contain at least one heteroatom selected from among N, O, S, optionally fused with another ring, with the proviso that these rings may contain one or more carbonyl or thiocarbonyl functional groups, the alkyl radical or the said rings being saturated or unsaturated and optionally substituted with at least one substituent A₂; A₁ is a halogen, a saturated or unsaturated, linear or branched C₁-C₂₂ alkyl radical, optionally substituted with one or more groups A₂, one of the groups CF₃, CN, OR₅, SR₅, NR₅R′₅, C(═NR₅)R′₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅, SiR₅R′₅R″₅, SiR₅(OSiR′₅R″₅R′″₅)OSiR′₅R″₅R′″₅, and NR₅R′₅R″₅R′″₅+ wherein R₅, R′₅, R″₅ and R′″₅, which may be identical or different, are each hydrogen, a linear or branched C₁-C₂₂ alkyl radical or a 4- to 7-atom ring member which may contain at least one heteroatom selected from among N, O, S, optionally fused with another ring, with the proviso that these rings may contain one or more carbonyl or thiocarbonyl functional groups, the alkyl radical or said rings being saturated or unsaturated and optionally substituted with at least one substituent A₂, a saturated or unsaturated 4- to 7-atom ring member optionally containing at least one heteroatom selected from among N, O and S, optionally substituted with one or more groups A₂, optionally fused with another ring or with another 3-(2-azacycloalkylidene)-1,3-dihydroindol-2-one compound, with the proviso that these rings may contain one or more carbonyl or thiocarbonyl functional groups, optionally positively charged; A₂ is a halogen, a saturated or unsaturated, linear or branched C₁-C₂₂ alkyl radical, one of the groups CF₃, CN, OR, SR, NRR′, C(═NR)R′, C(═NR)NR′R″, NRC(═NR′)NR″R′″, COR, CSR, COOR, CONRR′, NRCOR′, NRCONR′R″, CSNRR′, SO₂NRR′, NRSO₂R′, SO₂R, SiRR′R″, SiR(OSiR′R″R′″)OSiR′R″R′″, and NRR′R″R′″+ wherein R, R′, R″ and R′″, which may be identical or different, are each hydrogen or a linear or branched C₁-C₂₂ alkyl radical; y is 1, 2, 3 or 4; and x ranges from 1 to 2n+2, formulated into (b) a topically applicable, cosmetically/dermatologically acceptable carrier therefor.

2. The cosmetic/dermatological composition as defined by claim 1, said at least one 3-(2-azacycloalkylidene)-1,3-dihydroindol-2-one compound having one of the following formulae (II) or (III): 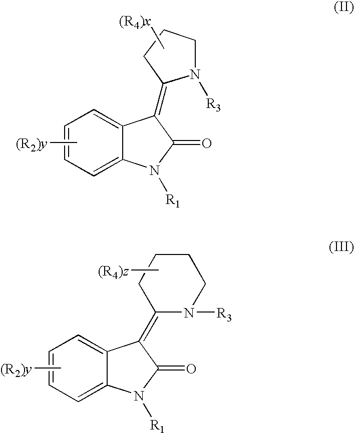
in which R₁ and R₃, which may be identical or different, are each hydrogen, a saturated or unsaturated, linear or branched C₁-C₈ alkyl radical, a phenyl radical, or a radical COR₅ or SO₂R₅ wherein R₅ is a saturated or unsaturated, linear or branched C₁-C₈ alkyl radical, or a phenyl radical; R₂ is hydrogen, a saturated or unsaturated, linear or branched C₁-C₈ alkyl radical, a phenyl radical, one of the radicals OR₅, NR₅′₅, NR₅COR′₅, COOR₅ and CONR₅R′₅ wherein R₅ and R′₅, which may be identical or different, are each hydrogen, a saturated or unsaturated, linear or branched C₁-C₈ alkyl radical, a phenyl radical, or the radical CF₃; R₄ is hydrogen, a saturated or unsaturated, linear or branched C₁-C₈ alkyl radical, a phenyl radical or a radical OR₅ wherein R₅ is hydrogen, a saturated or unsaturated, linear or branched C₁-C₈ alkyl radical, or a phenyl radical; and x, y and z are each 1, 2, 3 or 4.

3. The cosmetic/dermatological composition as defined by claim 2, comprising at least one compound of formula (II), wherein: R₂ is hydrogen, OR₅, NR₅COR′₅ or NR₅R′₅ wherein R₅ and R′₅, which may be identical or different, are each hydrogen, a saturated or unsaturated, linear or branched C₁-C₈ alkyl radical, or a phenyl radical; R₄ is hydrogen; and R₁ and R₃, which may be identical or different, are each hydrogen, a saturated or unsaturated, linear or branched C₁-C₈ alkyl radical, or a phenyl radical.

4. The cosmetic/dermatological composition as defined by claim 3, comprising at least one compound of formula (II) selected from the group consisting of 1-ethyl-3-(1-methylpyrrolidin-2-ylidene)-1,3-dihydroindol-2-one 1-ethyl-3-pyrrolidin-2-ylidene-1,3-dihydroindol-2-one and pyrrolidine-2-ylidene-1,3-dihydroindol-2-one.

5. The cosmetic/dermatological composition as defined by claim 1, said at least one 3-(2-azacycloalkylidene)-1,3-dihydroindol-2-one compound comprising from 0.01% to 20% by weight thereof.

6. The cosmetic/dermatological composition as defined by claim 1, said at least one 3-(2-azacycloalkylidene)-1,3-dihydroindol-2-one compound comprising from 0.1% to 10% by weight thereof.

7. The cosmetic/dermatological composition as defined by claim 1, formulated as an oil-in-water or water-in-oil emulsion.

8. The cosmetic/dermatological composition as defined by claim 1, further comprising at least one additional organic and/or inorganic UV-A and/or UV-B sunscreen.

9. The cosmetic/dermatological composition as defined by claim 8, comprising at least one additional organic sunscreen selected from the group consisting of anthranilates; cinnamic derivatives; dibenzoylmethane derivatives; salicylic derivatives, camphor derivatives; triazine derivatives; benzophenone derivatives; β,β-diphenyl acrylate derivatives; benzotriazole derivatives; benzalmalonate derivatives; benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene; 4,4-diarylbutadiene derivatives, and mixtures thereof.

10. The cosmetic/dermatological composition as defined by claim 9, said at least one additional organic sunscreen being selected from the group consisting of:
Ethylhexyl Salicylate,
Ethylhexyl Methoxycinnamate, Octocrylene,
Phenylbenzimidazole Sulphonic Acid,
Benzophenone-3,
Benzophenone-4,
Benzophenone-5,
n-Hexyl 2-(4-diethylamino-2-hydroxybenzoyl)benzoate,
4-Methylbenzylidene camphor,
Terephthalylidene dicamphor sulfonic acid,
Disodium Phenyl Dibenzimidazole Tetra-sulphonate,
2,4,6-Tris(diisobutyl 4′-aminobenzalmalonate)-s-triazine,
Anisotriazine,
Ethylhexyl triazone,
Diethylhexyl Butamido Triazone,
Methylene bis-Benzotriazolyl Tetramethylbutylphenol,
Drometrizole Trisiloxane,
Polysilicone-15
1,1-dicarboxy(2,2′-dimethylpropyl)-4,4-diphenylbutadiene
2,4-bis-[5-1(dimethylpropyl)benzoxazol-2-yl-(4-phenyl)-imino]-6-(2-ethylhexyl)-imino-1,3,5-triazine, and mixtures thereof.

11. The cosmetic/dermatological composition as defined in claim 8, comprising at least one additional inorganic sunscreen pigment or nanopigment of a metal oxide, coated or uncoated.

12. The cosmetic/dermatological composition as defined by claim 11, said at least one pigment or nanopigment comprising titanium, zinc, iron, zirconium and/or cerium oxides, and mixtures thereof, coated or uncoated.

13. The cosmetic/dermatological composition as defined by claim 1, further comprising at least one agent for artificially bronzing and/or tanning the skin.

14. The cosmetic/dermatological composition as defined by claim 1, further comprising at least one adjuvant or additive selected from the group consisting of fatty substances, organic solvents, ionic or nonionic thickeners, demulcents, humectants, antioxidants, moisturizers, desquamating agents, anti-free-radical agents, antipollution agents, antibacterials, anti-inflammatory agents, depigmenting agents, propigmenting agents, opacifiers, stabilizers, emollients, silicones, anti-foaming agents, insect repellents, perfumes, preservatives, anionic, cationic, nonionic, zwitterionic or amphoteric surfactants, substance P antagonists, substance CGRP antagonists, fillers, pigments, polymers, propellants, alkalinizing or acidifying agents, and mixtures thereof.

15. The cosmetic/dermatological composition as defined by claim 1, formulated as a nonionic vesicular dispersion, a cream, a milk, a gel, a gel cream, a suspension, a dispersion, an oil, a powder, a solid stick, a mousse or a spray.

16. The cosmetic/dermatological composition as defined by claim 1, formulated as a makeup.

17. The cosmetic/dermatological composition as defined by claim 1, formulated for protecting the hair against ultraviolet rays and in the form of a shampoo, a lotion, a gel, an emulsion or a nonionic vesicular dispersion.

18. A regime or regimen for photoprotecting the skin and/or hair against the damaging effects of UV radiation, comprising topically applying thereon an effective amount of the cosmetic/dermatological composition as defined by claim 1.

19. A regime or regimen for controlling the variation in the color of the skin due to UV radiation, comprising topically applying thereon an effective amount of the cosmetic/dermatological composition as defined by claim 1.

20. A synthetic polymer or glass containing an effective photostabilizing amount of at least one 3-(2-azacycloalkylidene)-1,3-dihydroindol-2-one compound as defined in claim 1.
